(19) United States
(12) Patent Application Publication
(10) Pub. No.: US 2014/0249417 A1
(43) Pub. Date: Sep. 4, 2014

(54) ULTRASOUND DIAGNOSTIC DEVICE AND ULTRASOUND DIAGNOSTIC DEVICE CONTROL METHOD

(71) Applicant: KONICA MINOLTA, INC., Chiyoda-ku, Tokyo (JP)

(72) Inventors: Junya Ookouchi, Tokyo (JP); Takao Suzuki, Tokyo (JP)

(73) Assignee: Konica Minolta, Inc., Chiyoda-ku, TOKYO (JP)

(21) Appl. No.: 14/349,645

(22) PCT Filed: Oct. 4, 2012
(86) PCT No.: PCT/JP2012/006396

§ 371 Date: Apr. 3, 2014

(30) Foreign Application Priority Data
Oct. 4, 2011 (JP) 2011-219749

(51) Int. Cl. A61B 8/00; A61B 8/14; A61B 8/08

(52) U.S. Cl. CPC A61B 8/54; A61B 8/0891; A61B 8/5223; A61B 8/463; A61B 8/14; A61B 8/5207
USPC 600/443; 600/449

(57) ABSTRACT

An ultrasound diagnostic device to which an ultrasound probe is connectable, measuring an IMT of a vascular wall in a carotid artery, including: a transmission unit supplying a transmission signal to the probe for causing ultrasound transmission along a longitudinal cross-section of the carotid artery; a reception unit receiving a signal based on a reflected ultrasound received by the probe, and generating a reception signal; a vascular feature calculation unit extracting position information from the reception signal, including at least one of: piece positions for vascular wall pieces; and a relative relationship between piece positions, and detecting a boundary between a CCA and a CCA bulb according to variation in the position information with respect to the longitudinal direction of the carotid artery; a ROI determination unit determining a ROI defining an IMT measurement range with respect to the boundary; and an IMT measurement unit measuring the IMT.

### TECHNICAL FIELD

The present disclosure pertains to an ultrasound diagnostic device and to a control method for the ultrasound diagnostic device, and particularly pertains to carotid artery diagnostic technology for early discovery of arteriosclerosis.

### BACKGROUND ART

In recent years, increasing numbers of patients are suffering from circulatory problems, including ischemia, cerebral infarction, and myocardial infarction. Early discovery and treatment of clinical signs for arteriosclerosis is important for preventing the above problems.

One indicator for diagnosing arteriosclerosis is a thickness measurement of the tunica intima—tunica media complex in the carotid artery (hereinafter also IMT, Intima-Media Thickness). Ultrasound exams have become widespread as a simple and non-invasive measurement method. IMT measurement is performed on the carotid artery because the carotid artery is a predilection site for arteriosclerosis, and because measurement of the carotid artery is easily performed with ultrasound waves at a comparatively shallow depth of 2 cm to 3 cm below the skin surface. Also, IMT measurement is performed according to a B-mode image, which as an ultrasound diagnostic image of a cross-section taken along the longitudinal direction of a blood vessel (hereinafter also termed a longitudinal cross-section).

FIG. 16 is a schematic diagram of a B-mode image representing a longitudinal cross-section of the carotid artery. As shown, a B-mode image 10 displays a blood vessel 11 where a vascular wall nearer to the ultrasound probe 100 is a near wall 12a and a vascular wall farther from the ultrasound probe 100 is a far wall 12b. The area between the near wall 12a and the far wall 12b is a lumen 12c in which blood flows. The blood vessel 11 is a carotid artery made up of a Common Carotid Artery (hereinafter also CCA) arranged in the central direction, as well as an Internal Carotid Artery (hereinafter also ICA) and an External Carotid Artery (hereinafter also ECA) arranged in the peripheral direction. The bulb of the common carotid artery (hereinafter simply termed bulb) is located between CCA and the ICA and ECA. The bifurcation of the common carotid artery (hereinafter, Bif) is found at the point where the ICA and ECA split off from the bulb.

IMT measurement is performed as follows. First, upon obtaining a B-mode image as shown in FIG. 16, a region of interest 13 (hereinafter also ROI 13) is determined so as to cross the vascular wall. Next, the boundary between the lumen and the tunica intima (hereinafter, Lumen-Intima or LI boundary) and the boundary between the tunica media and the tunica adventitia (hereinafter, Media-Adventitia or MA boundary) of the vascular wall in the ROI 13 are detected, and an IMT measurement range is defined for the vascular wall within the ROI 13. The IMT is then calculated from the distance between the LI boundary and the MA boundary. A recommended IMT measurement range is, for example, defined as starting from the boundary between the CCA and the bulb (hereinafter also CCA-bulb boundary 14) and extending along the far wall toward the CCA for a range of 1 cm, as described in Non-Patent Literature 1.

Operations for determining the ROI 13 that defines the IMT measurement range are complex when these operations must be performed manually. As such, technology has been proposed for simplifying such complex operations and enabling simpler IMT measurement, such as Patent Literature 1 and 2, in which ROI 13 determination is automated. For instance, in Patent Literature 1, intensity values are added and averaged for each pixel in a B-mode image of a blood vessel longitudinal cross-section obtained by transmitting and receiving an ultrasound beam. Vascular wall positions are then extracted by using an inflection point of intensity values in the ultrasound beam transmission direction, and the ultrasound diagnostic device detects the ROI 13 in the B-mode image. Also, Patent Literature 2 discloses a ultrasound diagnostic device that determines the ROI 13 by detecting a cardiac wall two-dimensionally by binarizing a brightness signal of a cardiac wall B-mode image.

### CITATION LIST

### Patent Literature

[Patent Literature 1]
Japanese Patent Application Publication No. 2010-119842

[Patent Literature 2]
Japanese Patent Application Publication No. 2002-125971

### Non-Patent Literature

[Non-Patent Literature 1]
Stein J H, et al., “ASE CONSENSUS STATEMENT: Use of Carotid Ultrasound to Identify Subclinical Vascular Disease and Evaluate Cardiovascular Disease Risk: A Consensus Statement from the American Society of Echocardiography Carotid Intima-Media Thickness Task Force, Endorsed by the Society for Vascular Medicine”, J Am Soc Echocardiogr. 2008; 21:93-111.

[Non-Patent Literature 2]
Research Group for Early Arteriosclerosis, “maxIMT Measurement”, [online], Sep. 9, 2010 (retrieved Sep. 30, 2011), URL: http://www.imt-ca.com/contents/e08.html

### SUMMARY OF INVENTION

### Technical Problem

However, the configurations of Patent Literature 1 and 2 are technology for determining a ROI that crosses the vascular wall but not for automatically determining a ROI that enables IMT measurement of the vascular wall in the longitudinal direction. However, with these methods, an operator must determine the ROI for the carotid artery in the longitudinal direction. As a result, measurements are difficult for an inexperienced operator to make, and the diagnostic time needed for precise measurement is longer.

In consideration of the above problem, the present invention aims to provide an ultrasound diagnostic device and an ultrasound diagnostic device control method where the ultrasound diagnostic device measures IMT of a carotid artery vascular wall by enabling swift IMT measurement through a simple operation that can be made by an inexperienced operator, via automatic determination of an ROI defining a measurement range for measuring the IMT.

### Solution to Problem

In order to achieve the above-described aim, the present disclosure provides an ultrasound diagnostic device to which an ultrasound probe is connectable, measuring an IMT of a vascular wall in a carotid artery, comprising: a transmission unit supplying a transmission signal to the ultrasound probe for causing the ultrasound probe to transmit an ultrasound along a longitudinal cross-section of the carotid artery; a reception unit receiving a signal based on a reflected ultrasound received by the ultrasound probe from the carotid artery, and generating a reception signal; a vascular feature calculation unit extracting position information from the reception signal, the position information including at least one of: piece positions for respective pieces making up the vascular wall of the carotid artery; and a relative relationship between the piece positions, and detecting a boundary between a common carotid artery and a common carotid artery bulb according to variation in the position information with respect to the longitudinal direction of the carotid artery; a ROI determination unit determining a ROI that defines a measurement range for measuring the IMT, with respect to the boundary; and an IMT measurement unit measuring the IMT of the vascular wall in the ROI.

Also, a control method for an ultrasound diagnostic device to which an ultrasound probe is connectable, measuring an IMT of a vascular wall in a carotid artery comprises: supplying a transmission signal to the ultrasound probe for causing the ultrasound probe to transmit an ultrasound along a longitudinal cross-section of the carotid artery; receiving a signal based on a reflected ultrasound received by the ultrasound probe from the carotid artery, and generating a reception signal; extracting position information from the reception signal, the position information including at least one of: piece positions for respective pieces making up the vascular wall of the carotid artery; and a relative relationship between the piece positions, and detecting a boundary between a common carotid artery and a common carotid artery bulb according to variation in the position information with respect to the longitudinal direction of the carotid artery; determining a ROI that defines a measurement range for measuring the IMT, with respect to the boundary; and measuring the IMT of the vascular wall in the ROI.

### Effects of Invention

The ultrasound diagnostic device pertaining to an aspect of the disclosure enables automatic determination of an ROI defining a measurement area for measuring the IMT of a carotid artery vascular wall, and thereby enables swift IMT measurement through a simple operation performable by an inexperienced operator.

### BRIEF DESCRIPTION OF DRAWINGS

FIG. 1 is a block diagram indicating the configuration of an ultrasound diagnostic device 200 pertaining to Embodiment 1.

FIG. 2 is a block diagram indicating the configuration of a vascular feature calculation unit 3 in the ultrasound diagnostic device 200 pertaining to Embodiment 1.

FIG. 3 is a block diagram indicating the configuration of an IMT measurement unit 5 in the ultrasound diagnostic device 200 pertaining to Embodiment 1.

FIG. 4 is a flowchart of operations pertaining to IMT measurement performed by the ultrasound diagnostic device 200 of Embodiment 1.

FIG. 5 schematically illustrates a vascular diameter of the carotid artery with respect to the longitudinal direction as calculated by the vascular diameter calculation unit 31 in portion (a), and schematically illustrates vascular diameter variation along central to peripheral direction of portion (a) in portion (b).

FIG. 6 is a block diagram indicating the configuration of a vascular feature calculation unit 15 in an ultrasound diagnostic device 201 pertaining to Embodiment 2.

FIG. 7 is a flowchart of operations pertaining to IMT measurement performed by the ultrasound diagnostic device 201 of Embodiment 2.

FIG. 8 schematically illustrates a vascular wall position of the carotid artery with respect to the longitudinal direction as calculated by the vascular feature calculation unit 15 of Embodiment 2 in portion (a), and schematically illustrates vascular wall position variation along central to peripheral direction of portion (a) in portion (b).

FIG. 9 is a block diagram indicating the configuration of a vascular feature calculation unit 16 in an ultrasound diagnostic device 202 pertaining to Embodiment 3.

FIG. 10 schematically illustrates, an amount of variation in the vascular diameter of the carotid artery with respect to the longitudinal direction as a pulsation magnitude calculated by the vascular wall pulsation calculation unit 34 in portion (a), and schematically illustrates vascular wall pulsation magnitude variation along a central to peripheral direction of portion (a) (i.e., along the arrow of portion (a) in portion (b).

FIG. 11 is a flowchart of operations pertaining to IMT measurement performed by the ultrasound diagnostic device 202 of Embodiment 3.

FIG. 12 is a block diagram indicating the configuration of an ultrasound diagnostic device 203 pertaining to Embodiment 4.

FIG. 13 is a block diagram indicating the configuration of a vascular feature calculation unit 18 in an ultrasound diagnostic device 203 pertaining to Embodiment 4.

FIG. 14 is a flowchart of operations pertaining to IMT measurement performed by the ultrasound diagnostic device 203 of Embodiment 4.

FIG. 15 schematically illustrates the IMT of the carotid artery with respect to the longitudinal direction, as calculated by the IMT measurement unit 17 of Embodiment 4, in portion (a), and schematically illustrates IMT variation along central to peripheral direction of portion (a) in portion (b).

FIG. 16 is a schematic diagram of a B-mode image representing a longitudinal cross-section of the carotid artery.

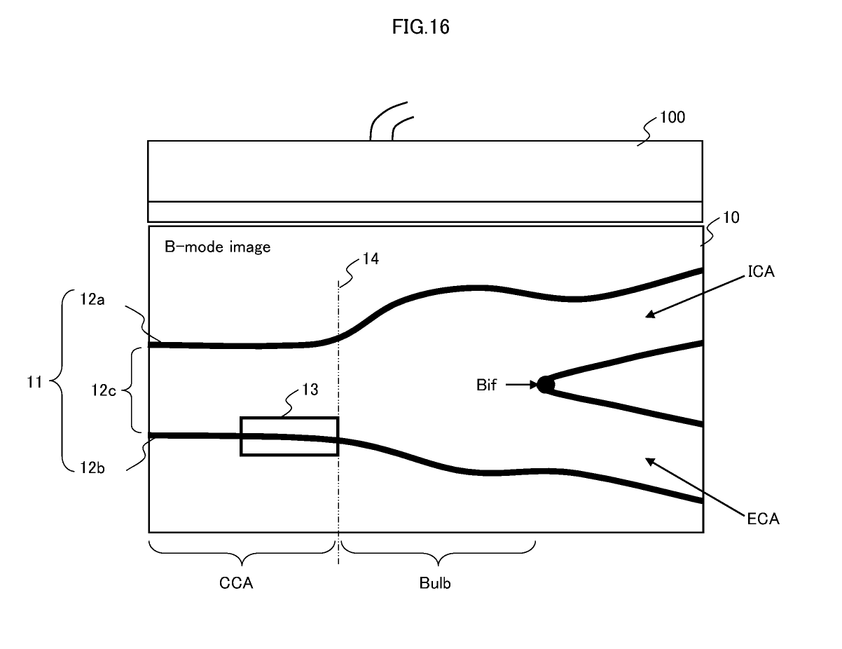

### DESCRIPTION OF EMBODIMENTS

<Background Leading to Embodiments>

Ultrasound diagnostic devices are used in various tests for determining a ROI 13 defining an IMT measurement range. For example, Non-Patent Literature 2 describes a testing method for the CCA-bulb boundary 14. There, the CCA-bulb boundary 14 is determined by representing the CCA-bulb boundary 14 as the inflection point of the vascular wall where the peripheral end of the CCA morphs into the bulb. The inflection point is then used to find an intersection of lines respectively extending from the CCA side and the bulb side of the boundary between the tunica adventitia and tunica media in the vicinity of the transition from CCA to bulb, and that intersection is identified as the CCA-bulb boundary 14.

The inventors have used the CCA-bulb boundary 14 identified using a detection method as described in Non-Patent Literature 2 as a basis for dedicated investigation into the realisation of ROI 13 determination for defining the IMT measurement range. For instance, the inventors debated whether or not the predetermined measurement range recommended in Non-Patent Literature 1 is usable for defining the ROI 13.

However, the method of Non-Patent Literature 2 is not able to detect the CCA-bulb boundary 14 for certain subjects. In these cases, the IMT measurement range cannot be automatically detected, thus requiring that the operator determine the IMT measurement range. Accordingly, the CCA-bulb boundary 14 detection described in Non-Patent Literature 2 was thought inapplicable as a method for determining the IMT measurement range, even if automated.

The inventors then considered the causes. The method of Non-Patent Literature 2 is able to detect the CCA-bulb boundary 14 when the B-mode image is obtained for a carotid artery that approximates an ideal shape. However, when a B-mode image for a carotid artery having an idiosyncratic shape is used, the CCA-bulb boundary 14 cannot be detected, and thus the IMT measurement range cannot be determined. For instance, when the subject's carotid artery has no curve in the vascular wall of the CCA-bulb boundary 14, detecting the inflection point becomes difficult. Also, despite the CCA-bulb boundary 14 being present in the subject's carotid artery, the inflection point is difficult to detect when the shape is difficult to observe using an ultrasound diagnostic device, and when the curvature of the neck during observation is such that the inflection point of the CCA-bulb boundary 14 cannot be observed. As a result, the CCA-bulb boundary 14 cannot be detected. For example, the above applies when at least one of the front wall and back wall of the bulb is flat, such that the inflection point of the CCA-bulb boundary 14 along the vascular wall is difficult to detect along said wall. Also, when a B-mode image is obtained in which the inflection point is difficult to observe, further operations must be repeatedly performed in order to obtain a B-mode image in which the inflection point is visible. This has been a problem in diagnosis when the inflection point remains ultimately undetectable.

In order for the ultrasound diagnostic device measuring the IMT of the vascular wall in the carotid artery to automatically determine the ROI 13 defining the measurement range of the IMT, the CCA-bulb boundary 14 must be detected regardless of the subject's carotid artery shape. Establishing a scanning method enabling such detection would be beneficial. Furthermore, IMT measurement requires that measurements be performed in the same measurement range each time in order to obtain an accurate measurement for diagnosis of the progress over a predetermined period of measurement.

As such, upon dedicated investigation, the inventors arrived at an ultrasound diagnostic device according to one aspect of the present disclosure, using a method of detecting the CCA-bulb boundary 14 that does not depend on the condition of the B-mode image or on the shape of the subject's carotid artery.

An ultrasound diagnostic device and an ultrasound diagnostic device control method pertaining to the Embodiments are described below, with reference to the accompanying drawings.

<Overview of Embodiments>

In one aspect, an ultrasound diagnostic device to which an ultrasound probe is connectable, measuring an IMT of a vascular wall in a carotid artery, comprises: a transmission unit supplying a transmission signal to the ultrasound probe for causing the ultrasound probe to transmit an ultrasound along a longitudinal cross-section of the carotid artery; a reception unit receiving a signal based on a reflected ultrasound received by the ultrasound probe from the carotid artery, and generating a reception signal; a vascular feature calculation unit extracting position information from the reception signal, the position information including at least one of: piece positions for respective pieces making up the vascular wall of the carotid artery; and a relative relationship between the piece positions, and detecting a boundary between a common carotid artery and a common carotid artery bulb according to variation in the position information with respect to the longitudinal direction of the carotid artery; a ROI determination unit determining a ROI that defines a measurement range for measuring the IMT, with respect to the boundary; and an IMT measurement unit measuring the IMT of the vascular wall in the ROI.

Here, position information includes at least one of a piece of information indicating a piece position for each piece of a vascular wall in the carotid artery, and a piece of information indicating a relative relationship between the piece positions of the vascular wall. The piece position for each piece of the vascular wall is a position as seen in a cross-section of the vascular wall, for instance, indicating a position of the lumen-intima boundary, a position of the media-adventitia boundary, a position of the tunica adventitia circumference, and so on. The relative relationship between the pieces of the vascular wall is a positional relationship as seen in the cross-section of the vascular wall. For example, this may indicate an inner diameter of the blood vessel as represented by the distance between positions of the lumen-intima boundary, an inner diameter of the blood vessel as represented by the distance between positions of the tunica adventitia circumference, the IMT as represented by the distance between positions of the lumen-intima boundary and the media-adventitia boundary, the thickness of the vascular wall as represented by the distance between positions of the lumen-intima boundary and the tunica adventitia circumference, and so on.

In another aspect, the vascular feature calculation unit further includes a central-peripheral determination unit determining a central direction and a peripheral direction for the carotid artery according to the position information, and the ROI determination unit determines the ROI according to the peripheral direction and the central direction for the carotid artery.

In an alternate aspect, the position information is a vascular diameter indicating one of: distance between a near-wall lumen-intima boundary position and a far-wall lumen-intima boundary position; distance between a near-wall media-adventitia boundary position and a far-wall media-adventitia boundary position; and distance between a near-wall tunica adventitia circumference position and a far-wall tunica adventitia outer boundary position.

In a further aspect, the position information pertains to at least one of a lumen-intima boundary position, a media-adventitia boundary position, and a tunica adventitia circumference position.

In yet another aspect, the vascular feature calculation unit detects the boundary according to variation in one of the lumen-intima boundary position, the media-adventitia boundary position, and the tunica adventitia circumference position at a common location along the longitudinal direction of the carotid artery, using a plurality of frames acquired from the reception signal within a predetermined period.

In still another aspect, the vascular wall thickness is the IMT as represented by distance between a lumen-intima boundary position and a media-adventitia boundary position.

In yet a further aspect, the ultrasound diagnostic device further comprises a display; and an image formation unit generating a B-mode image signal based on the reception signal, for displaying a B-mode image on the display, wherein the vascular feature calculation unit extracts the position information from the B-mode image signal.

Also, in another alternate aspect, the position information is represented in coordinates used when displaying the B-mode image on the display.

In yet another aspect, a control method for an ultrasound diagnostic device to which an ultrasound probe is connectable, measuring an IMT of a vascular wall in a carotid artery, comprising: supplying a transmission signal to the ultrasound probe for causing the ultrasound probe to transmit an ultrasound along a longitudinal cross-section of the carotid artery; receiving a signal based on a reflected ultrasound received by the ultrasound probe from the carotid artery, and generating a reception signal; extracting position information from the reception signal, the position information including at least one of: piece positions for respective pieces making up the vascular wall of the carotid artery; and a relative relationship between the piece positions, and detecting a boundary between a common carotid artery and a common carotid artery bulb according to variation in the position information with respect to the longitudinal direction of the carotid artery; determining a ROI that defines a measurement range for measuring the IMT, with respect to the boundary; and measuring the IMT of the vascular wall in the ROI.

### Embodiment 1

An ultrasound diagnostic device pertaining to Embodiment 1 is described below, with reference to the accompanying drawings.

### <Configuration>

(General Configuration)

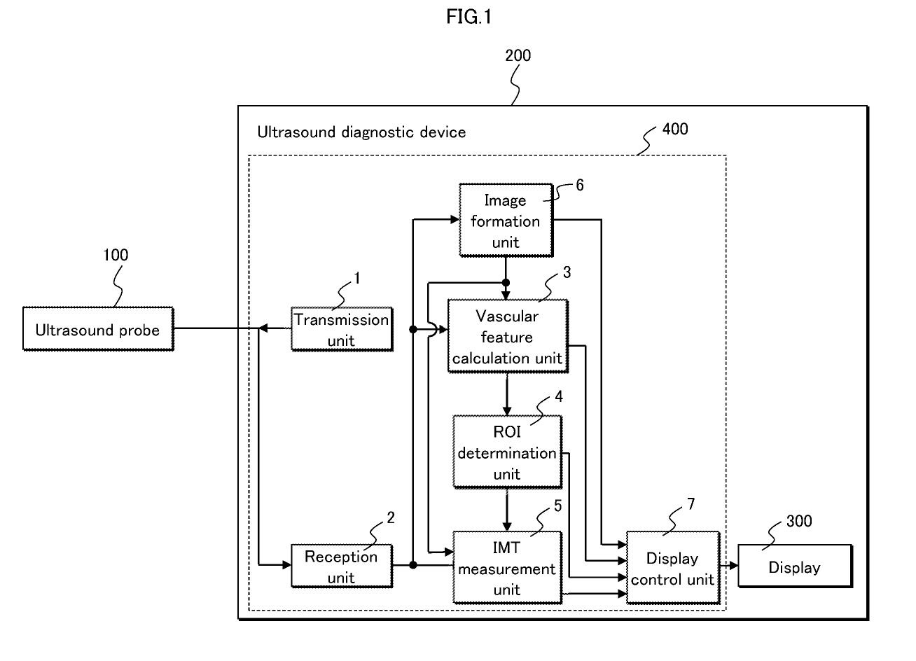

FIG. 1 is a block diagram indicating the configuration of an ultrasound diagnostic device 200 pertaining to Embodiment 1. The ultrasound diagnostic device 200 is configured to be electrically connectable to an ultrasound probe 100 transmitting an ultrasound. FIG. 1 illustrates the ultrasound probe 100 as connected to the ultrasound diagnostic device 200. The ultrasound diagnostic device 200 includes a controller 400 and a display 300. The controller 400 includes a transmission unit 1, a reception unit 2, a vascular feature calculation unit 3, a ROI determination unit 4, an IMT measurement unit 5, an image formation unit 6, and a display control unit 7.

(Transmission Unit 1)

The transmission unit 1 generates an electronic signal, in pulse or continuous form, for causing the ultrasound probe 100 to transmit an ultrasound, and performs a transmission process of providing the electronic signal to the ultrasound probe 100 as a transmission signal.

(Ultrasound Probe 100)

The ultrasound probe 100 includes a transducer array in which a plurality of piezoelectric elements are arranged into multiple columns. The ultrasound probe 100 converts the transmission signal, which is the electronic signal provided in pulse or continuous form by the transmission unit 1, into an ultrasound beam in pulse or continuous form, and when the transducer array is in contact with the skin surface of the subject, fires the ultrasound beam toward the carotid artery from the skin surface. In order to obtain a B-mode image of the carotid artery in long-axis cross-section, the ultrasound probe 100 is arranged such that the transducer array is parallel to the longitudinal direction of the carotid artery when the ultrasound beam is fired. The ultrasound probe 100 then receives an ultrasound echo signal, which is a reflected ultrasound returned from the subject, then converts the ultrasound echo signal into an electronic signal through the transducer array and provides the electronic signal to the reception unit 2. The electronic signal is used to convert the amplitude of the echo signal into a voltage value.

(Reception Unit 2)

The reception unit 2 performs a reception process of amplifying the electronic signal received from the ultrasound probe 100 and performing analogue-to-digital (hereinafter, A/D) conversion to generate a reception signal. The reception signal is then supplied to the vascular feature calculation unit 3, the IMT measurement unit 5, and the image formation unit 6. The reception signal is, for example, made up of a plurality of signals obtained along the transducer array direction and in a depth direction oriented away from the transducer array. Each of these is a digital signal obtained by performing A/D conversion on an electronic signal converted from the amplitude of an echo signal.

(Image Formation Unit 6)

The image formation unit 6 generates B-mode image data based on the reception signal that depicts the carotid artery, and supplies the data to the vascular feature calculation unit 3, the IMT measurement unit 5, and the display control unit 7. The B-mode image data are an image signal in which coordinate conversion has mainly been applied to the reception signal in order to correspond with the Cartesian coordinate system used for display on a screen of the display 300.

(Vascular Feature Calculation Unit 3)

The vascular feature calculation unit 3 analyses vascular shape features based on the reception signal or the B-mode image data to detect the CCA-bulb boundary 14. Information pertaining to the detected CCA-bulb boundary 14 is then provided to the ROI determination unit 4 and the display control unit 7.

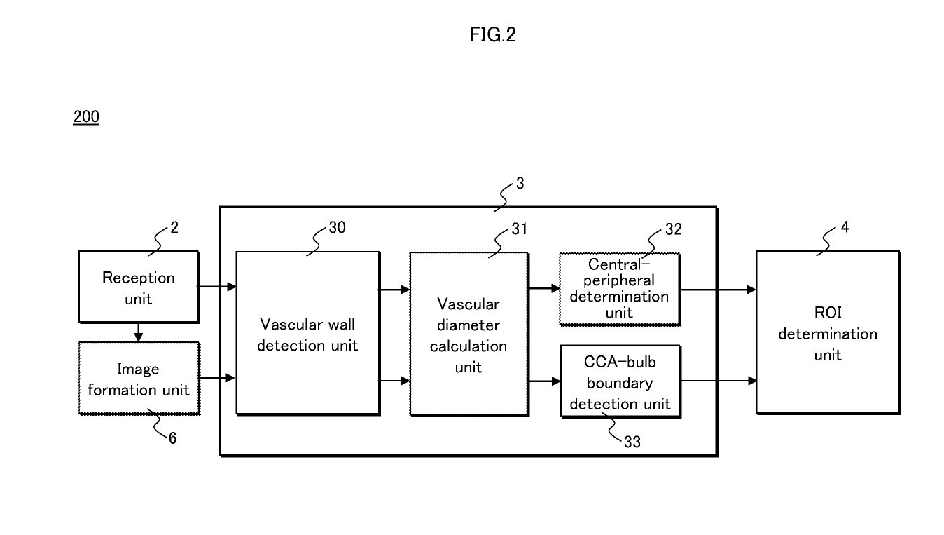

FIG. 2 is a block diagram indicating the configuration of the vascular feature calculation unit 3 in the ultrasound diagnostic device 200 pertaining to Embodiment 1. As shown, the vascular feature calculation unit 3 includes a vascular wall detection unit 30, a vascular diameter calculation unit 31, a central-peripheral determination unit 32, and a CCA-bulb boundary detection unit 33.

The vascular wall detection unit 30 extracts a vascular wall from the reception signal of the reception unit 2 or from the B-mode image data of the image formation unit 6, and detects a coordinate position of the vascular wall within the B-mode image. Specifically, position information indicating a position for each portion making up the vascular wall of the carotid artery is extracted from the reception signal generated by the reception unit 2 to detect the coordinate position of display in the B-mode image on the display 300. Alternatively, the position information indicating the position for each portion making up the vascular wall of the carotid artery may be directly extracted from the B-mode image data generated by the image formation unit 6 to detect the coordinate position of display in the B-mode image on the display 300. The position information for the vascular wall is extracted along the longitudinal direction of the carotid artery and used to calculate variation along the longitudinal direction. The variation along the longitudinal direction in the position information for the vascular wall represents vascular features of the carotid artery.

The vascular diameter calculation unit 31 calculates a vascular diameter as the distance between a coordinate position of a near wall and a coordinate position of a far wall based on the coordinate position of the vascular wall detected by the vascular wall detection unit 30. Information for the vascular diameter is extracted along the longitudinal direction of the carotid artery and used to calculate variation with respect to the longitudinal direction. The variation in the position information with respect to the longitudinal direction for the vascular diameter represents vascular features of the carotid artery.

The central-peripheral determination unit 32 makes a determination of central direction and peripheral direction according to the coordinate position for the vascular wall detected by the vascular wall detection unit 30. That is, the variation with respect to the longitudinal direction of the position information for the vascular wall in the reception signal or in the B-mode image data is used to calculate which end, with respect to the longitudinal direction, is in the central direction and which end is in the peripheral direction. Specifically, the distance between the coordinate position of the near wall and the coordinate position of the far wall gradually increases along the longitudinal direction when the blood vessel is gradually expanding from the CCA to the bulb. This indicates correspondence with the peripheral direction.

The CCA-bulb boundary detection unit 33 detects the CCA-bulb boundary 14 according to the vascular diameter variation along the longitudinal direction as calculated by the vascular diameter calculation unit 31. The method of detection is described later.

Although not illustrated in FIG. 2, the information obtained by the central-peripheral determination unit 32 and the CCA-bulb boundary detection unit 33 is also supplied to the display control unit 7.

(ROI Determination Unit 4)

The ROI determination unit 4 determines an appropriate position of the ROI 13 defining a predetermined measurement range for IMT measurement, according to the CCA-bulb boundary 14 and the central and peripheral direction information received from the vascular feature calculation unit 3. The ROI determination unit 4 then supplies the position information determined for the ROI 13 to the IMT measurement unit 5 and to the display control unit 7. For instance, the ROI 13 may be determined as beginning at the position of the CCA-bulb boundary 14 and covering a range of 1 cm toward the CCA along the far wall, according to the information pertaining to the central and peripheral direction and the position information of the CCA-bulb boundary 14. The range indicated in Non-Patent Literature 1, cited above, is definable as the ROI 13.

(IMT Measurement Unit 5)

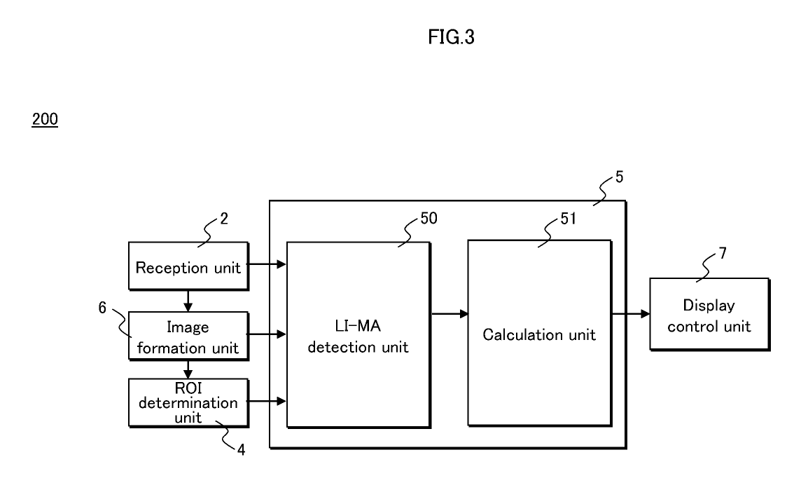

FIG. 3 is a block diagram indicating the configuration of the IMT measurement unit 5 in the ultrasound diagnostic device 200 pertaining to Embodiment 1. As shown, the IMT measurement unit 5 includes a LI-MA detection unit 50 and a calculation unit 51. Specifically, the LI-MA detection unit 50 detects respective positions of the LI boundary and MA boundary in the vascular wall of the carotid artery based on the signal in the ROI 13 within the reception signal or the B-mode image data. A spacing between the respective positions of the LI and MA boundaries is measured by the calculation unit 51 as the IMT. The detection method used for finding the LI boundary and MA boundary positions to measure the IMT is based on a publicly-known method. A method based on signal intensity waveform of the reception signal, such as the method described in International Patent Application Publication No. 2007/108359, is applicable to detecting the respective LI and MA boundaries.

Also, the calculation unit 51 calculates an IMT value as the maximum thickness (hereinafter also maxIMT) or mean thickness (hereinafter also mean IMT) of the IMT in the ROI 13 according to the LI and MA boundaries detected by the LI-MA detection unit 50.

(Display Control Unit 7)

The display control unit 7 displays information pertaining to the CCA-bulb boundary 14 supplied by the vascular feature calculation unit 3, ROI 13 position information supplied by the ROI determination unit 4, IMT measurement results supplied by the IMT measurement unit 5, and B-mode image data supplied by the image formation unit 6 on the display 300.

<Operations>

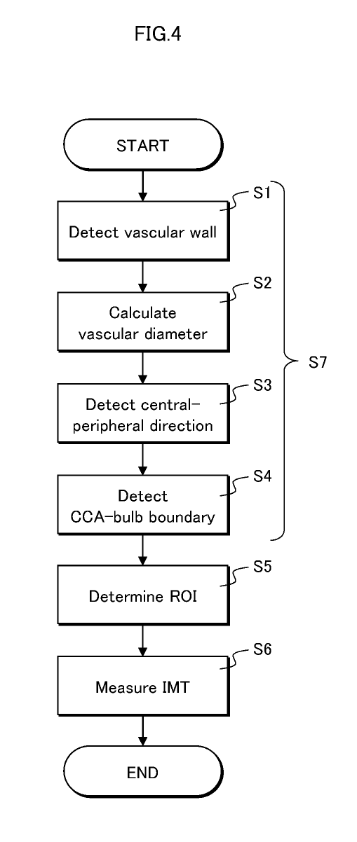

The operations of the ultrasound diagnostic device 200 configured as described above are described with reference to the flowchart of FIG. 4. The flowchart of FIG. 4 indicates operations pertaining to IMT measurement performed by the ultrasound diagnostic device 200 of Embodiment 1. The transmission and reception of the ultrasound beam to the subject having the carotid artery are performed by typical methods, and explanations thereof are thus omitted. That is, the operations of automatically determining the ROI 13 and of measuring the IMT within the determined ROI 13 are described.

(Step 1 (S1))

In step 1 (S1), the vascular feature calculation unit 3 extracts the vascular wall based on the reception signal supplied by the reception unit 2 and on the B-mode image data supplied by the image formation unit 6, then detects the respective coordinate positions of each part of the vascular wall as displayed in the B-mode image. The parts of the vascular wall represent indicate coordinate positions of the LI boundary and the MA boundary at the near and far walls of the carotid artery.

Specifically, the vascular wall detection unit 30 performs smoothing by applying a low-pass filter as pre-processing of the reception signal supplied by the reception unit 2 or the B-mode image data supplied by the image formation unit 6. Afterward, derivatives of the reception signal or the B-mode image data are taken with respect to the depth direction of the subject to whom the ultrasound beam has been transmitted. The differentiated values are extracted as maxima and minima indicating respective positions of the near wall and the far wall. The coordinate position of the near wall and the far wall are then extracted. Position information is extracted from the reception signal or the B-mode image data concerning the lumen-intima boundary, the media-adventitia boundary, and the circumference of the vascular wall, the latter being the outermost portion of the blood vessel. The position of the boundary between the CCA and the bulb is then detected according to position information variation with respect to the longitudinal direction of the carotid artery. Given that the tunica intima and tunica media of the vascular wall are easily deformable under the influence of the pulse, and that appropriate vascular position cannot be detected when plaque has formed in the lumen, extracting the media-adventitia boundary of the circumference of the vascular wall as the vascular wall is beneficial in that these issues have less effect.

(Step 2 (S2))

In step 2 (S2), the vascular diameter calculation unit 31 calculates the vascular diameter at each position along the longitudinal direction of the blood vessel by taking the difference between the respective coordinate positions of the near and far walls, as detected by the vascular wall detection unit 30.

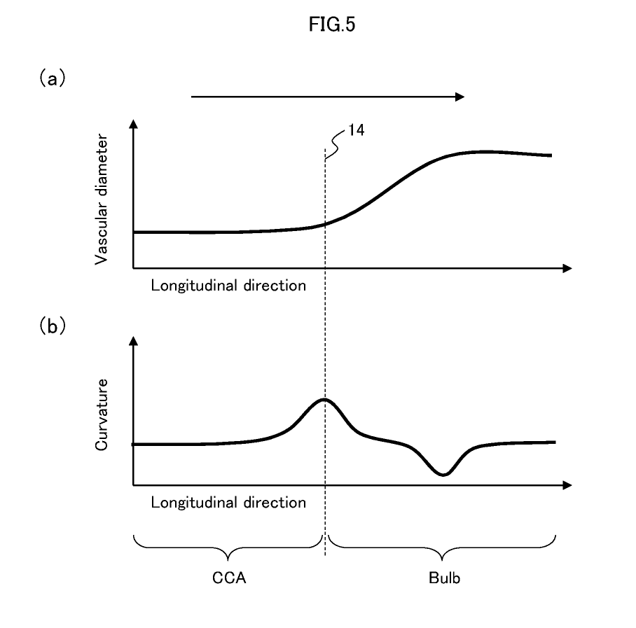

Specifically, the distance is calculated with respect to a plurality of positions of the far wall, using the coordinate position of the near wall as reference. A shortest distance among these compute distances is calculated as the vascular diameter. This is performed for each position of the near wall in the longitudinal direction, to calculate the vascular diameter at each position along the longitudinal direction of the blood vessel. This method enables correct vascular diameter calculation, despite the blood vessel displayed in the B-mode image being curved. The distance between the near wall and the far wall may also be calculated with reference to a coordinate position of the far wall. Portion (a) of FIG. 5 schematically illustrates a vascular diameter of the carotid artery with respect to the longitudinal direction as calculated by the vascular diameter calculation unit 31. Portion (b) of FIG. 5 schematically illustrates vascular diameter variation from the central to the peripheral direction of portion (a) (i.e., the direction of the arrow in portion (a)). In portion (a), the vertical axis represents vascular diameter and the horizontal axis represents the longitudinal direction.

(Step 3 (S3))

In step 3 (S3), the central-peripheral determination unit 32 determines the central and peripheral directions of the carotid artery displayed in the B-mode image from the vascular diameter at each position with respect to the longitudinal direction as calculated by the vascular diameter calculation unit 31. That is, the direction where the vascular diameter is largest is the peripheral direction given that the bulb has a greater vascular diameter than the CCA. Accordingly, detecting the direction in which the vascular diameter is largest based on the variation waveform obtained in step 2 as shown in portion (a) of FIG. 5 enables making the central-peripheral direction determination.

(Step 4 (S4))

In step 4 (S4), the CCA-bulb boundary detection unit 33 detects the CCA-bulb boundary 14 according to the variation waveform of the vascular diameter taken along the longitudinal direction in step 2 (S2) (FIG. 5, portion (a)). The vascular diameter of the CCA is mostly stable along the longitudinal direction. In contrast, the vascular diameter of the bulb, which is roughly spherical, suddenly increases as the CCA transitions into the bulb. Accordingly, and as shown in portion (a) of FIG. 5, the onset portion, where the vascular diameter suddenly begins to increase, is the CCA-bulb boundary 14. Thus, the CCA-bulb boundary 14 is made detectable.

Furthermore, the variation waveform shown in portion (b) of FIG. 5 is obtained by taking the second derivative of the variation waveform indicated in portion (a) of FIG. 5. The CCA-bulb boundary 14 is also detectable as the maximum thereof. Accordingly, the increase onset portion is further clarified, enabling easier specification thereof.

The above-described steps 1-4 (S1-S4) jointly form a vascular feature calculation step 7 (S7).

(Step 5 (S5))

In step 5 (S5), the ROI determination unit 4 determines the ROI 13 according to the central and peripheral direction determined in step 3 (S3) and the CCA-bulb boundary 14 determined in step 4 (S4). For example, a range of 1 cm on the far wall extending centrally from the detected CCA-bulb boundary 14 is beneficially determined as the ROI 13. As such, the ROI 13 is made to correspond to the range of measurement recommended in Non-Patent Literature 1.

(Step 6 (S6))

In step 6 (S6), the IMT measurement unit 5 measures the IMT in the ROI 13 determined in step 5. The calculation unit 51 measures the IMT as the distance between the respective positions of the LI boundary and the MA boundary in the vascular wall of the carotid artery detected in step 1 (S1). Afterward, the measurement results are displayed on the display 300 and so on, thereby completing one set of IMT measurement operations for the ultrasound diagnostic device 200.

<Effects>

According to the above configuration, the ultrasound diagnostic device 200 pertaining to Embodiment 1 detects the CCA-bulb boundary 14 where no curvature is present in the longitudinal direction of the vascular diameter, even in cases where, for example, the B-mode image shows a large curvature in the longitudinal direction from the CCA to the bulb. Thus, the CCA-bulb boundary 14 is detectable irrespective of the shape of the carotid artery in the subject, and automatic detection of the CCA-bulb boundary is made possible with greater precision. As such, the ultrasound diagnostic device 200 measuring the IMT of the carotid artery vascular wall is able to automatically determine the ROI 13 for defining the measurement range in which the IMT is actually measured. As a result, the IMT measurement is made at a more precise position or in an automatically-determined range, enabling accurate and quick IMT measurement to be performed by someone with less experience.

### Embodiment 2

### Configuration

(General Configuration)

An ultrasound diagnostic device 201 pertaining to Embodiment 2 differs from the ultrasound diagnostic device 200 of Embodiment 1 in that the vascular feature calculation unit 3 is replaced by a vascular feature calculation unit 15 detecting a boundary position between the common carotid artery CCA and the common carotid artery bulb based on variation in the vascular wall with respect to the longitudinal direction. The ultrasound diagnostic device 200 of Embodiment 1 detects the CCA-bulb boundary 14 based on variations in the vascular diameter in the vicinity of the CCA-bulb boundary 14. In Embodiment 2, the CCA-bulb boundary 14 is detected using variations in the coordinate position of the vascular wall. Components of the ultrasound diagnostic device 201 other than the vascular feature calculation unit 15 are configured as indicated by the block diagram of FIG. 1 pertaining to the ultrasound diagnostic device 200. Explanations thereof are thus omitted.

(Vascular Feature Calculation Unit 15)

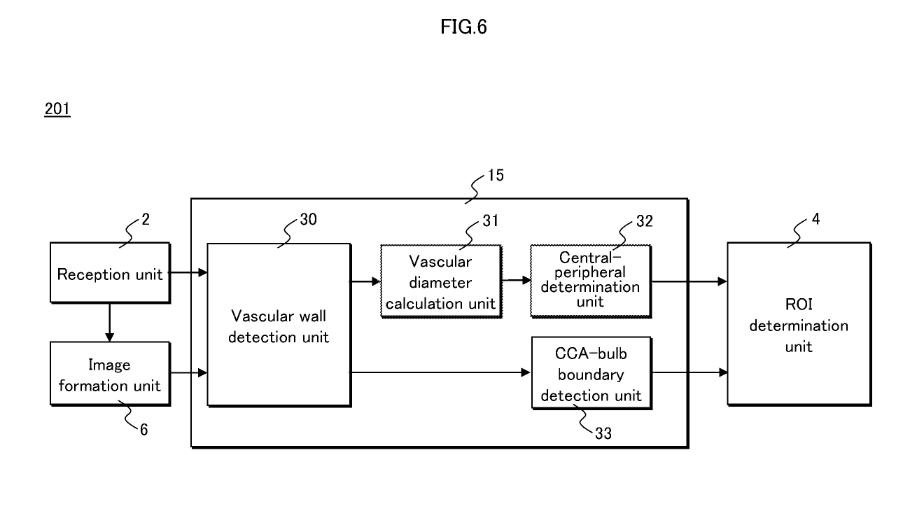

FIG. 6 is a block diagram indicating the configuration of the vascular feature calculation unit 15 in the ultrasound diagnostic device 201 pertaining to Embodiment 2. As shown, the vascular feature calculation unit 15 includes the vascular wall detection unit 30, the vascular diameter calculation unit 31, the central-peripheral determination unit 32, and the CCA-bulb boundary detection unit 33. Among these, the vascular wall detection unit 30, the vascular diameter calculation unit 31, and the central-peripheral determination unit 32 are configured as described in Embodiment 1. As such, explanations thereof are omitted.

A CCA-bulb boundary detection unit 33 detects the CCA-bulb boundary 14 based on the coordinate position of the vascular wall in the B-mode image detected by the vascular wall detection unit 30. That is, the vascular feature calculation unit 15 extracts position information from the reception signal or the B-mode image data concerning the lumen-intima boundary, the media-adventitia boundary, and the circumference of the vascular wall, the latter being the outermost portion of the blood vessel. The position of the boundary between the CCA and the bulb is then detected according to position information variation with respect to the longitudinal direction of the carotid artery. Given that the tunica intima and tunica media of the vascular wall are easily deformable under the influence of the cardiac pulse, and that appropriate vascular position cannot be detected when plaque has formed in the lumen, extracting the media-adventitia boundary of the circumference of the vascular wall as the vascular wall is beneficial in that these issues have less effect.

<Operation>

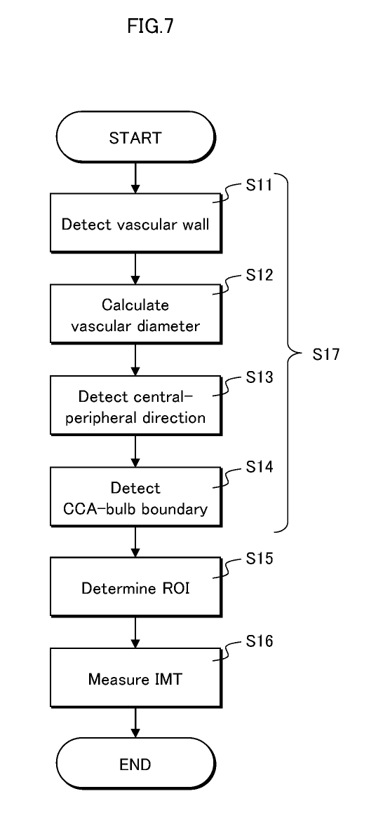

The operations of the ultrasound diagnostic device 201 pertaining to Embodiment 2 and configured as described above are described with reference to FIG. 7. FIG. 7 is a flowchart of operations pertaining to IMT measurement performed by the ultrasound diagnostic device 201 of Embodiment 2. As with Embodiment 1, the operations of automatically determining the ROI 13 and of measuring the IMT within the determined ROI 13 are described.

(Step 11 (S11))

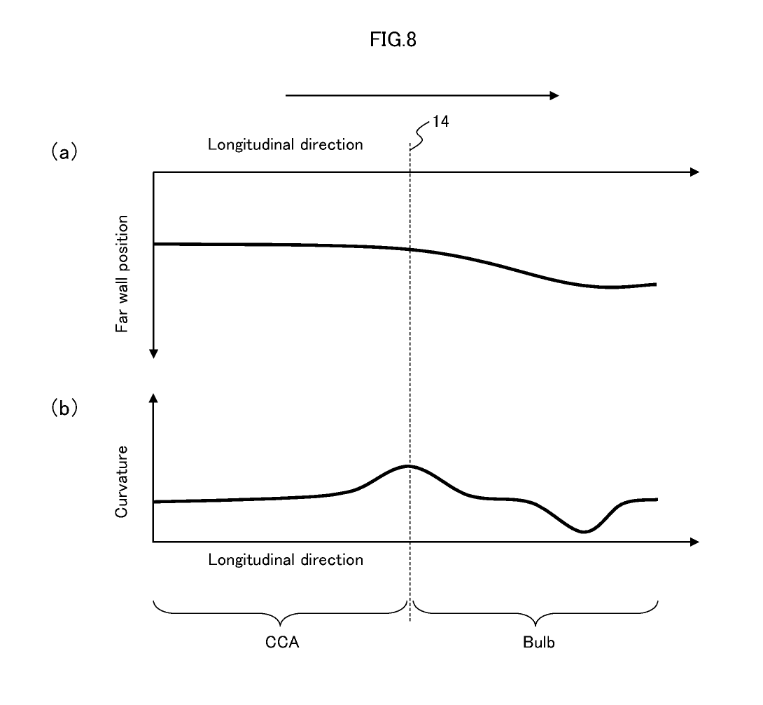

In step 11 (S11), the vascular feature calculation unit 15 extracts the vascular wall based on the reception signal supplied by the reception unit 2 and the B-mode image data supplied by the image formation unit 6, then detects the respective coordinate positions of each part of the vascular wall as displayed in the B-mode image. Explanations of this process identical are omitted where identical to step 1 (S1) of Embodiment 1. The point of difference from step 1 (S1) is that a variation waveform of the coordinate position with respect to the longitudinal direction is extracted from at least one extracted coordinate position of the near wall and the far wall. Portion (a) of FIG. 8 schematically illustrates vascular wall positions with respect to the longitudinal direction of the carotid artery as calculated by the vascular feature calculation unit 15 of Embodiment 2. Portion (b) of FIG. 8 schematically illustrates vascular wall position variation from the central to the peripheral direction of portion (a) (i.e., the direction of the arrow in portion (a)). The vascular wall position used is beneficially one or more pieces of position information among position information concerning the lumen-intima boundary, the media-adventitia boundary, and the circumference of the vascular wall, the latter being the outermost portion of the blood vessel.

When the tunica intima and tunica media of the vascular wall are easily deformable under the influence of the pulse and when plaque has formed on the lumen, as in Embodiment 1, extracting the position media-adventitia boundary of the circumference of the vascular wall as the vascular wall is beneficial in that these issues have less effect.

(Step 12 (S12) and Step 13 (S13))

Steps 12 (S12) and 13 (S13) are respectively identical to steps 2 (S2) and 3 (S3) of Embodiment 1. As such, explanations thereof are omitted.

Steps 12 (S12) and 13 (S13) involve determinations made for determining, in the later-described ROI 13 determination (step 15 (S15)), a measurement range or determining a direction for position measurement based on the CCA-bulb boundary detected in step 14 (S14). Accordingly, when taking the IMT measurement position as the CCA-bulb boundary 14, there is no need to make the central-peripheral direction determination. As such, steps 12 (S12) and 13 (S13) are not needed, nor are the corresponding components, namely the vascular diameter calculation unit 31 and the central-peripheral determination unit 32.

(Step 14 (S14))

In step 14 (S14), the CCA-bulb boundary detection unit 33 detects the CCA-bulb boundary 14 based on the variation waveform (see portion (a) of FIG. 8) with respect to the longitudinal direction obtained in step 11 (S11). The CCA-bulb boundary 14 is detected by noting a position at which the vascular diameter suddenly increases as the CCA transitions into the bulb. This point is the same as Embodiment 1. The point of difference from Embodiment 1 is that the vascular wall appearing in the B-mode image is used instead of the vascular diameter. That is, the CCA-bulb boundary 14 is detected as the onset of the sudden increase in the variation waveform of the vascular wall coordinate position. Accordingly, and as shown in portion (a) of FIG. 8, the portion where the vascular wall position suddenly begins to change is the CCA-bulb boundary 14. Thus, the CCA-bulb boundary 14 is made detectable.

Furthermore, the variation waveform shown in portion (b) of FIG. 8 is obtained by taking the second derivative of the variation waveform indicated in portion (a) of FIG. 8. The CCA-bulb boundary 14 is also detectable as the maximum thereof. Accordingly, the increase onset portion is further clarified, enabling easier specification thereof.

The above-described steps 11-14 (S11-S14) jointly form a vascular feature calculation step 17 (S17).

(Step 15 (S15) and Step 16 (S16))

Steps 15 (S15) and 16 (16) are identical to steps 5 (S5) and 6 (S6) of Embodiment 1. Explanations thereof are thus omitted.

As described above, the ultrasound diagnostic device 201 pertaining to Embodiment 2 is able to detect the CCA-bulb boundary 14 according to variations in the vascular wall coordinate position, given that little variation occurs in the vascular diameter in the vicinity of the CCA-bulb boundary 14 while variation in vascular diameter does occur at the CCA.

### Embodiment 3

### Configuration

(General Configuration)

An ultrasound diagnostic device 202 pertaining to Embodiment 3 differs from the ultrasound diagnostic device 200 of Embodiment 1 in that the vascular feature calculation unit 3 is replaced by a vascular feature calculation unit 16 detecting a boundary position between the common carotid artery CCA and the common carotid artery bulb based on an amount of variation in the vascular diameter as caused by vascular pulsation. The amount of vascular diameter variation caused by vascular pulsation itself varies from central to peripheral areas, and is large at the border of the CCA-bulb boundary 14 vicinity. As such, the ultrasound diagnostic device 202 pertaining to Embodiment 3 detects the vascular diameter variation caused by pulsation along the longitudinal direction, and detects the CCA-bulb boundary 14 by finding the onset of increase the amount of variation. Components of the ultrasound diagnostic device 202 other than the vascular feature calculation unit 16 are configured as indicated by the block diagram of FIG. 1 pertaining to the ultrasound diagnostic device 200. Explanations thereof are thus omitted.

(Vascular Feature Calculation Unit 16)

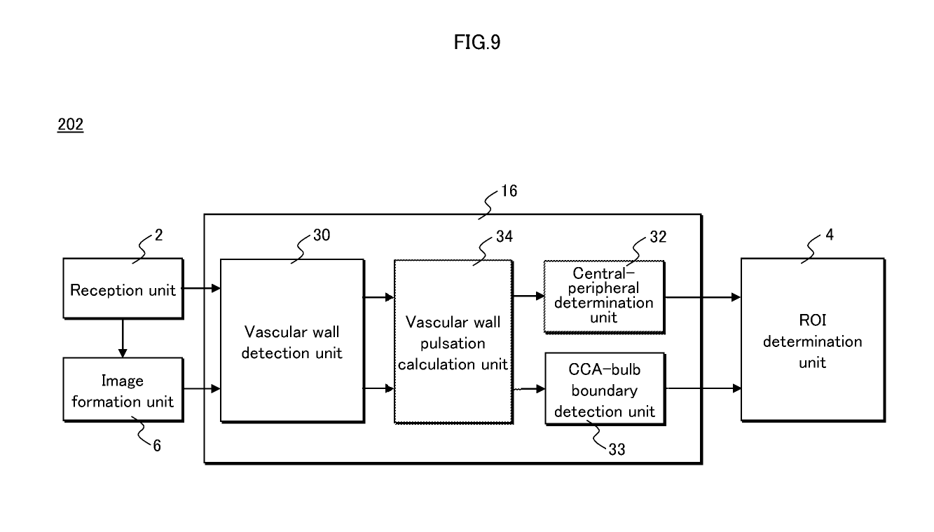

The following describes the configuration of the vascular feature calculation unit 16 with reference to the accompanying drawings. FIG. 9 is a block diagram indicating the configuration of the vascular feature calculation unit 16 in the ultrasound diagnostic device 202 pertaining to Embodiment 3. The point of difference from Embodiment 1 is that the vascular diameter calculation unit 31 has been replaced by a vascular wall pulsation calculation unit 34. Other components are identical to those of Embodiment 1.

The vascular wall pulsation calculation unit 34 detects motion of the vascular wall based on the reception signal or B-mode image data in a plurality of frames within a fixed interval. For example, the motion of the vascular wall is beneficially detected based on the reception signal or B-mode image data in a plurality of frames within a fixed interval of one pulsation cycle.

Specifically, a plurality of coordinate positions are first detected for the vascular wall in one frame of the reception signal or B-mode image data. This process is performed on the reception signal or B-mode image data for a plurality of frames in one pulsation cycle. Variations between the coordinate position at each vascular wall position are then detected. A maximum point of the variation between coordinate positions in each frame is detected as the amount of vascular wall displacement. Furthermore, an amount variation in the vascular diameter along the longitudinal direction is obtained from a variation waveform of the vascular wall displacement with respect to the longitudinal direction. That is, the amount of vascular diameter variation at each position along the longitudinal direction of the vessel is calculated by computing the difference between the near wall coordinate position and the far wall coordinate position as detected by the vascular wall detection unit 30.

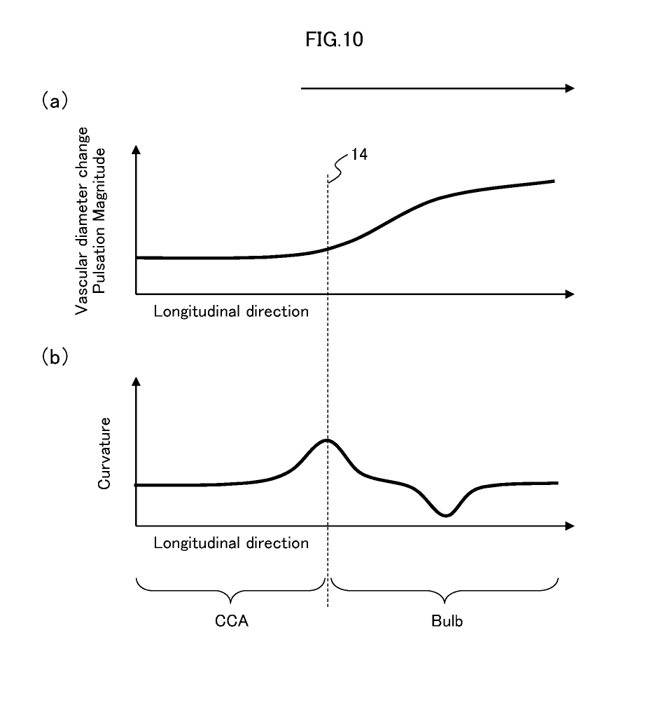

A waveform as indicated in portion (a) of FIG. 10 is obtained from the amount of vascular diameter variation at each position. Portion (a) of FIG. 10 schematically illustrates the magnitude of vascular diameter variation as the magnitude pulsations with respect to the longitudinal direction of the carotid artery as calculated by the vascular wall pulsation calculation unit 34 of Embodiment 3. Portion (b) of FIG. 10 schematically illustrates vascular wall variation with respect to vascular pulsation from the central to the peripheral direction of portion (a) (i.e., the direction of the arrow in portion (a)).

<Operation>

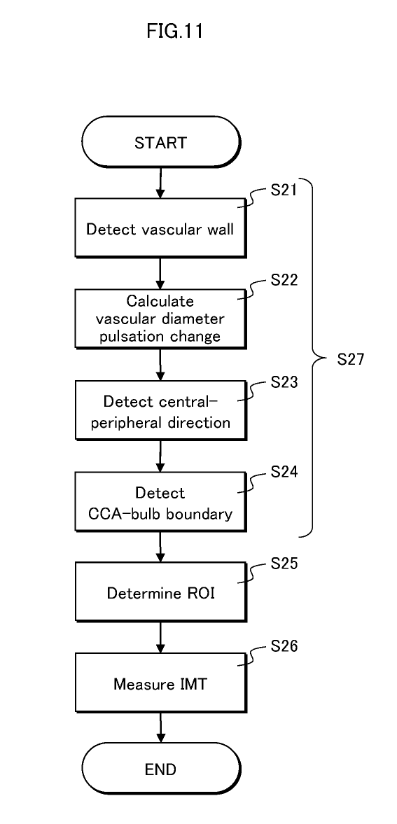

The operations of the ultrasound diagnostic device 202 configured as described above are described with reference to the drawings. FIG. 11 is a flowchart of operations pertaining to IMT measurement performed by the ultrasound diagnostic device 202 of Embodiment 3. As with Embodiment 1, the operations of automatically determining the ROI 13 and of measuring the IMT within the determined ROI 13 are described.

(Step 21 (S21))

In step 21 (S21), the vascular feature calculation unit 16 extracts the vascular wall according to the reception signal supplied by the reception unit 2 and the B-mode image data supplied by the image formation unit 6, then detects the positions of each part of the vascular wall as displayed in the B-mode image. Explanations of this process identical are omitted where identical to step 1 (S1) of Embodiment 1. The point of difference from step 1 (S1) is that the vascular wall position is detected from the reception signal or B-mode image data for a plurality of frames.

(Step 22 (S22))

In step 22 (S22), the vascular wall pulsation calculation unit 34 finds a plurality of vascular wall and detects these coordinate positions in a plurality of frames based on the corresponding reception signal or B-mode image data. The distance between coordinate positions at maximum difference between coordinate positions for a plurality of frames is then calculated for each of the vascular positions, and the vascular wall displacement with respect to the longitudinal direction is detected as a variation waveform. Furthermore, an amount variation in the vascular diameter along the longitudinal direction is obtained from a variation waveform of the vascular wall displacement with respect to the longitudinal direction. That is, as indicated by portion (a) of FIG. 10, the amount of vascular diameter variation along the longitudinal direction of the vessel is calculated by computing the difference between the near wall coordinate position and the far wall coordinate position as detected by the vascular wall detection unit 30.

(Step 23 (S23))

Step 23 (S23) involves detecting the central-peripheral direction. The variant method described for step 3 (S3) of Embodiment 1 is applied. That is, the central and peripheral directions are detected by tracking coordinate position variation in the vascular wall based on the reception signal or B-mode image data for a plurality of frames.

(Step 24 (S24))

In step 24 (S24), the CCA-bulb boundary detection unit 33 detects the CCA-bulb boundary 14 at the onset of sudden expansion of the variation waveform, based on the variation waveform obtained in step 22 (S22) (see portion (a) of FIG. 10). Furthermore, the variation waveform shown in portion (b) of FIG. 10 is obtained by taking the second derivative of the variation waveform indicated in portion (a) of FIG. 10. The CCA-bulb boundary 14 is also detectable as the maximum thereof. Accordingly, the increase onset portion is further clarified, enabling easier specification thereof.

The above-described steps 21-24 (S21-S24) jointly form a vascular feature calculation step 27 (S27).

(Step 25 (S25) and Step 26 (S26))

Afterward, the process proceeds to steps 25 (S25) and 26 (S26). However, these are identical to equivalent steps in Embodiment 1 and the explanations thereof are thus omitted.

Thus, as described above, the ultrasound diagnostic device 202 pertaining to Embodiment 3 uses the point at which the vascular diameter variation increases due to vascular pulsation in the vicinity of the CCA-bulb boundary 14 to detect the CCA-bulb boundary 14.

### Embodiment 4

### Configuration

(General Configuration)

An ultrasound diagnostic device 203 pertaining to Embodiment 4 differs from the ultrasound diagnostic device 200 of Embodiment 1 in that the vascular feature calculation unit 3 is replaced by a vascular feature calculation unit 18 detecting the CCA-bulb boundary 14 according to a vascular wall IMD value. The vascular wall thickness suddenly increases when the CCA-bulb boundary 14 transitions into the bulb. As such, the ultrasound diagnostic device 203 pertaining to Embodiment 4 detects the vascular wall thickness variation along the longitudinal direction, and detects the CCA-bulb boundary 14 by finding the onset of increase the vascular wall thickness. The thickness of the vascular wall may be derived from the boundary between the tunica intima and the lumen, as well as the boundary between the tunica adventitia and extra-vascular tissues. However, in Embodiment 3, the IMT value is used as representing a correlation with vascular wall thickness.

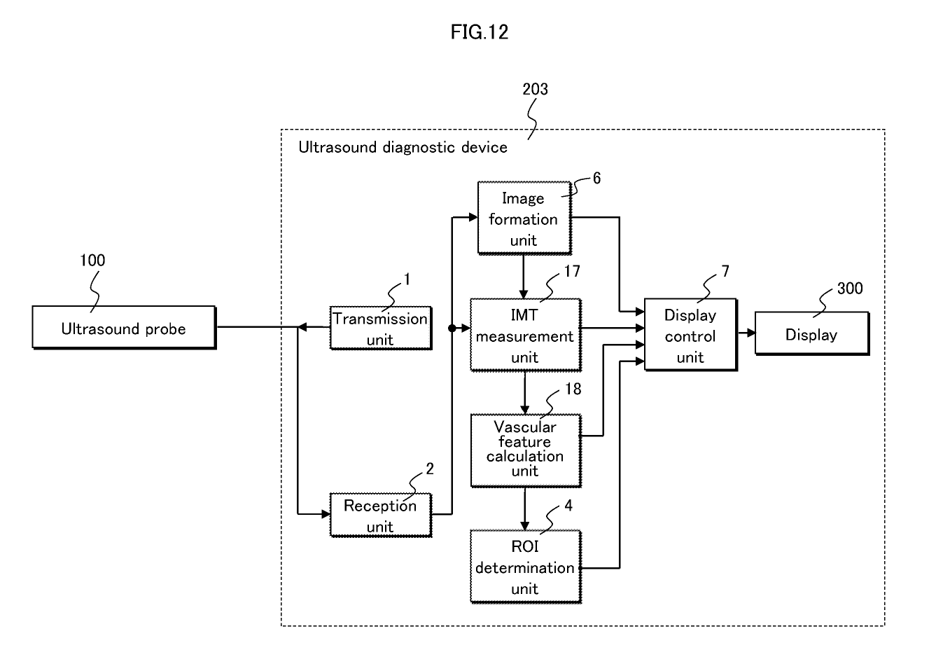

FIG. 12 is a block diagram indicating the configuration of an ultrasound diagnostic device 203 pertaining to Embodiment 4. Components of the ultrasound diagnostic device 203 of Embodiment 4 other than the IMT measurement unit 17 and the vascular feature calculation unit 18 are configured as indicated by the block diagram of FIG. 1 pertaining to the ultrasound diagnostic device 200. Explanations thereof are thus omitted.

(IMT Measurement Unit 17)

The IMT measurement unit 17 analyses the reception signal or B-mode image data, extracts the LI boundary and the MA boundary of the carotid artery, and performs IMT measurement based on the coordinate positions thereof. A variation waveform of the IMT value with respect to the vascular longitudinal direction is then obtained.

(Vascular Feature Calculation Unit 18)

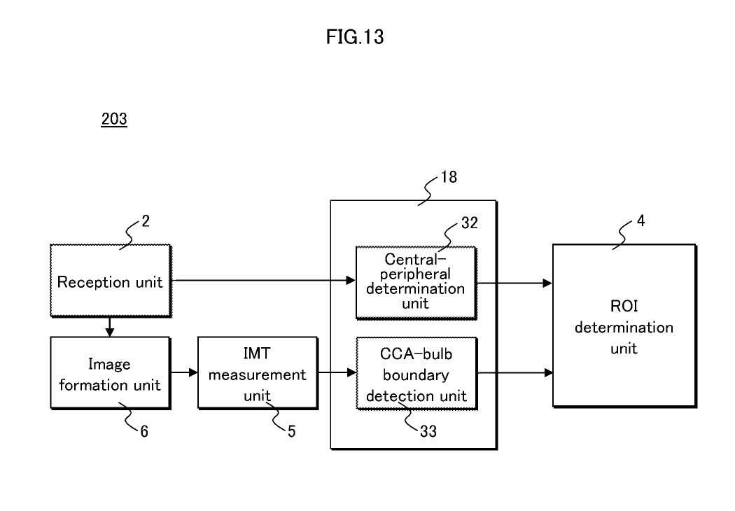

FIG. 13 is a block diagram indicating the configuration of the vascular feature calculation unit 18 in the ultrasound diagnostic device 203 pertaining to Embodiment 4. As shown in FIG. 13, the vascular feature calculation unit 18 includes the central-peripheral determination unit 32 and the CCA-bulb boundary detection unit 33, and detects the CCA-bulb boundary 14 as well as the central and peripheral directions from the IMT value calculated by the IMT measurement unit 5.

<Operation>

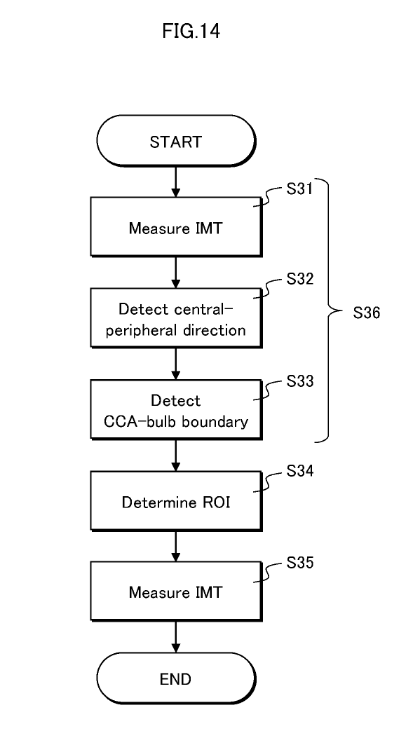

The operations of the ultrasound diagnostic device 203 configured as described above are described with reference to the drawings. FIG. 14 is a flowchart of operations pertaining to IMT measurement performed by the ultrasound diagnostic device 203 of Embodiment 4. As with Embodiment 1, the operations of automatically determining the ROI 13 and of measuring the IMT within the determined ROI 13 are described.

(Step 31 (S31))

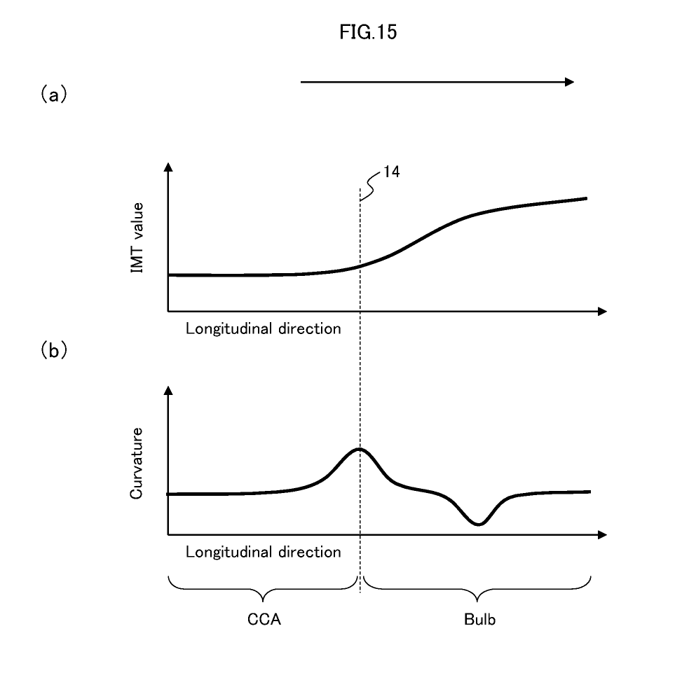

In step 31 (S31), the IMT value is measured at a plurality positions of the carotid artery based on the reception signal or B-mode image data for a plurality of frames in a predetermined interval. For example, the motion of the vascular wall is beneficially detected based on the reception signal or B-mode image data in a plurality of frames within a fixed interval of a single pulsation cycle. A variation waveform of the IMT value with respect to the longitudinal direction of the carotid artery is then obtained. Portion (a) of FIG. 15 schematically illustrates IMT measured with respect to the longitudinal direction of the carotid artery as calculated by the IMT measurement unit 17 of Embodiment 4. Portion (b) of FIG. 15 schematically illustrates IMT variation from the central to the peripheral direction of portion (a) (i.e., the direction of the arrow in portion (a)).

Calculating the IMT value based on the reception signal or B-mode image data for one frame is sufficient for detecting the CCA-bulb boundary 14. In step 31 (S31), IMT values are calculated using the reception signal or B-mode image data for a plurality of frames so as to use the values for determining the central-peripheral direction in the later-described step 32 (S32).

(Step 32 (S32))

In step 32 (S32), the central-peripheral direction is detected. This detection is performed according to the IMT values. The IMT values for one pulsation are such that the value increases when the vessel contracts and decreases when the vessel expands. Accordingly, IMT values for a predetermined vascular wall position are detected in the reception signal or B-mode image data for a plurality of frames, thus enabling the central-peripheral direction to be determined from IMT value variation between frames.

(Step 33 (S33))

In step 33 (S33), the CCA-bulb boundary detection unit 33 detects the CCA-bulb boundary 14 as the onset of a sudden increase in the variation waveform, based on the variation waveform (see portion (a) of FIG. 15) obtained in step 31 (S31). Furthermore, the variation waveform shown in portion (b) of FIG. 15 is obtained by taking the second derivative of the variation waveform indicated in portion (a) of FIG. 15. The CCA-bulb boundary 14 is also detectable as the maximum thereof. Accordingly, the increase onset portion is further clarified, enabling easier specification thereof.

The above-described steps 31-33 (S31-S33) jointly form a vascular feature calculation step 36 (S36).

(Step 34 (S34) and Step 35 (S35))

Afterward, the process advances to steps 34 (S34) and 35 (S35). However, these are identical to steps 5 (S5) and 6 (S6) in Embodiment 1 and the explanations thereof are thus omitted.

As described above, the ultrasound diagnostic device 203 pertaining to Embodiment 4 uses the increase in vascular wall thickness in the vicinity of the CCA-bulb boundary 14 to detect the CCA-bulb boundary 14.

<Variations>

Although the ultrasound diagnostic device has been described according to the above Embodiments, no such limitation is intended. The following variations may also be applied to the ultrasound diagnostic device.

(1) In step 1 (S1), step 11 (S11), and step 21 (S21), the vascular wall detection unit 30 of the vascular feature calculation unit 3, 15, and 16 performs smoothing by applying a low-pass filter as pre-processing of the reception signal supplied by the reception unit 2 or the B-mode image data supplied by the image formation unit 6. Afterward, derivatives of the reception signal or the B-mode image data are taken with respect to the depth direction of the subject to whom the ultrasound beam has been transmitted. The differentiated values are extracted as maxima and minima indicating respective positions of the near wall and the far wall. However, other methods may also be applied to the above-described vascular wall detection example. For instance, pre-processing may be applied using a planarization filter, a media filter, or similar to change the weighting of peripheral pixels. Any filter intended for smoothing may be applied to the configuration. Also, edge enhancement may be applied by performing binarization. In addition, vascular detection may be performed using correlations. Furthermore, differences in tissue elasticity and blood flow regions may also be used for detection. Detection of a vascular position using a plurality of frames enables the reduction of noise and errors.
(2) In step 3 (S3), step 13 (S13), step 23 (S23), and step 33 (S33), the central-peripheral determination unit 32 enables determination of the central and peripheral directions of the vascular wall by detecting a direction of pulse wave propagation using the propagation of the pulse wave from the central direction to the peripheral direction in accordance with vascular pulsation. That is, in order to obtain chronological variation in the vascular wall due to vascular pulsation, the central and peripheral directions may be detected by tracking coordinate position variation in the vascular wall based on the reception signal or B-mode image data for a plurality of frames.

Furthermore, the central-peripheral determination unit 32 enables determination of the central and peripheral directions from the blood flow direction, given that blood flows from the central direction toward the peripheral direction. In such a case, a Doppler function or similar may be used for blood flow detection.

(3) Step 5 (S5), step 15 (S15), step 25 (S25), and step 34 (S34) determine the ROI 13 as a predetermined position and measurement range or as a measurement position in one of the central direction and the peripheral direction, according to the CCA-bulb boundary 14 detected in step 4 (S4), step 14 (S14), step 24 (S24, and step 33 (S33). However, the position of the CCA-bulb boundary 14 may also be used as the IMT measurement position. In such a case, there is no need to determine the central direction and the peripheral direction. Thus, the central-peripheral determination unit 32 used in step 3 (S3), step 13 (S13), step 23 (S23), and step 32 (S32) is not needed.

<Conclusion>

As described above, Embodiment 1 detects the CCA-bulb boundary 14 based on vascular diameter variation with respect to the longitudinal direction in turn based on vascular wall coordinate positions with respect to the longitudinal direction of the carotid artery, Embodiment 2 detects the CCA-bulb boundary 14 according to vascular wall coordinate position variation with respect to the longitudinal direction of the carotid artery, Embodiment 3 detects the CCA-bulb boundary 14 based on an amount of variation in vascular diameter caused by pulsation with respect to the longitudinal direction of the carotid artery, and Embodiment 4 detects the CCA-bulb boundary 14 based on vascular wall thickness with respect to the longitudinal direction of the carotid artery.

As such, the ultrasound diagnostic device 200 measuring the IMT of the carotid artery vascular wall is able to automatically determine the ROI 13 for defining the measurement range in which the IMT is actually measured. In particular, when the CCA-bulb boundary 14 has no inflection point, or when the carotid artery is of a shape that is difficult to observe, accurate automatic determination of the measurement position and range for measuring the IMT are enabled. As a result, the IMT measurement is made at a more precise position or in an automatically-determined range, enabling accurate and quick IMT measurement to be performed with simple operations by someone with less experience.

<Supplement>

Each of the Embodiments described above is a non-limiting Embodiment of the device of the present disclosure. The quantities, shapes, materials, components, arrangement position and connections between components, steps, order of steps, and so on listed in the Embodiments are given as examples, no limitation being intended thereby. Also, the components listed in the Embodiments and steps not listed as independent aspects representing highest-order concepts of the disclosure are described as optional components for a beneficial Embodiment.

Furthermore, in drawings referred to in the above Embodiments, in order to facilitate understanding, configuration elements are not necessarily illustrated to scale. The present disclosure is not limited by the above Embodiments, and appropriate modifications may be made so long as such modifications do not cause deviation from the general concept of the present disclosure.

Furthermore, the ultrasound diagnostic device is configured as circuit elements, lead lines, and so on disposed on a substrate. However, the electrical connections and circuits thereof are technological matter widely known from ultrasound diagnostic apparatus technology and the like and are applicable to various configurations. As such, explanations thereof are omitted as not being directly relevant to the disclosure. The above-described drawings are schematics that do not necessarily closely conform to reality.

### INDUSTRIAL APPLICABILITY

The present disclosure is widely applicable to an ultrasound diagnostic device and an ultrasound diagnostic device control method enabling automatic determination of a ROI for defining a measurement range in which to measure IMT of a carotid artery vascular wall, and enabling IMT measurement to be quickly performed by an inexperienced operator through simple operations.

### REFERENCE SIGNS LIST

1 Transmission unit
2 Reception unit
3, 15, 16, 18 Vascular feature calculation unit
4 ROI determination unit
5, 17 IMT measurement unit
6 Image formation unit
7 Display control unit
10 B-mode image
11 Blood vessel
12a Near wall
12b Far wall
13 ROI (Region Of Interest)
14 CCA-bulb boundary
30 Vascular wall detection unit
31 Vascular diameter calculation unit
32 Central-peripheral determination unit
33 CCA-bulb boundary detection unit
34 Vascular wall pulsation calculation unit
50 LI-MA detection unit
51 Calculation unit
100 Ultrasound probe
200, 201, 202, 203 Ultrasound diagnostic device
300 Display
400 Controller

1. An ultrasound diagnostic device to which an ultrasound probe is connectable, measuring an IMT of a vascular wall in a carotid artery, comprising:
a transmission unit supplying a transmission signal to the ultrasound probe for causing the ultrasound probe to transmit an ultrasound along a longitudinal cross-section of the carotid artery;
a reception unit receiving a signal based on a reflected ultrasound received by the ultrasound probe from the carotid artery, and generating a reception signal;
a vascular feature calculation unit extracting position information from the reception signal, the position information including at least one of: piece positions for respective pieces making up the vascular wall of the carotid artery; and a relative relationship between the piece positions, and detecting a boundary between a common carotid artery and a common carotid artery bulb according to variation in the position information with respect to the longitudinal direction of the carotid artery;
a ROI determination unit determining a ROI that defines a measurement range for measuring the IMT, with respect to the boundary; and
an IMT measurement unit measuring the IMT of the vascular wall in the ROI.

2. The ultrasound diagnostic device of claim 1, wherein
the vascular feature calculation unit further includes a central-peripheral determination unit determining a central direction and a peripheral direction for the carotid artery according to the position information, and
the ROI determination unit determines the ROI according to the peripheral direction and the central direction for the carotid artery.

3. The ultrasound diagnostic device of claim 1, wherein
the position information is a vascular diameter indicating one of:
distance between a near-wall lumen-intima boundary position and a far-wall lumen-intima boundary position;
distance between a near-wall media-adventitia boundary position and a far-wall media-adventitia boundary position; and
distance between a near-wall tunica adventitia circumference position and a far-wall tunica adventitia outer boundary position.

4. The ultrasound diagnostic device of claim 1, wherein
the position information pertains to at least one of a lumen-intima boundary position, a media-adventitia boundary position, and a tunica adventitia circumference position.

5. The ultrasound diagnostic device of claim 4, wherein
the vascular feature calculation unit detects the boundary according to variation in one of the lumen-intima boundary position, the media-adventitia boundary position, and the tunica adventitia circumference position at a common location along the longitudinal direction of the carotid artery, using a plurality of frames acquired from the reception signal within a predetermined period.

6. The ultrasound diagnostic device of claim 1, wherein
the position information is a vascular wall thickness.

7. The ultrasound diagnostic device of claim 6, wherein
the vascular wall thickness is the IMT as represented by distance between a lumen-intima boundary position and a media-adventitia boundary position.

8. The ultrasound diagnostic device of claim 1, further comprising:
a display; and
an image formation unit generating a B-mode image signal based on the reception signal, for displaying a B-mode image on the display, wherein
the vascular feature calculation unit extracts the position information from the B-mode image signal.

9. The ultrasound diagnostic device of claim 8, wherein
the position information is represented in coordinates used when displaying the B-mode image on the display.

10. A control method for an ultrasound diagnostic device to which an ultrasound probe is connectable, measuring an IMT of a vascular wall in a carotid artery, comprising:
supplying a transmission signal to the ultrasound probe for causing the ultrasound probe to transmit an ultrasound along a longitudinal cross-section of the carotid artery;
receiving a signal based on a reflected ultrasound received by the ultrasound probe from the carotid artery, and generating a reception signal;
extracting position information from the reception signal, the position information including at least one of: piece positions for respective pieces making up the vascular wall of the carotid artery; and a relative relationship between the piece positions, and detecting a boundary between a common carotid artery and a common carotid artery bulb according to variation in the position information with respect to the longitudinal direction of the carotid artery;
determining a ROI that defines a measurement range for measuring the IMT, with respect to the boundary; and
measuring the IMT of the vascular wall in the ROI.
